(19) United States
(12) Patent Application Publication
(10) Pub. No.: US 2010/0167991 A1
(43) Pub. Date: Jul. 1, 2010

(54) Polysaccharides comprising carboxyl functional groups substituted by a hydrophobic alcohol derivative

(75) Inventors: Remi Soula, Lyon (FR); Olivier Soula, Meyzieu (FR); Gerard Soula, Meyzieu (FR); Richard Charvet, Rillieux la Pape (FR)

Correspondence Address: OLIFF & BERRIDGE, PLC, P.O. BOX 320850, ALEXANDRIA, VA, 22320-4850, US

(73) Assignee: ADOCIA, Lyon (FR)

(21) Appl. No.: 12/588,149

(22) Filed: Oct. 6, 2009

Related U.S. Application Data
(60) Provisional application No. 61/136,816, filed on Oct. 6, 2008.

(30) Foreign Application Priority Data
Oct. 6, 2008 (FR) 08 05506

(51) Int. Cl. A61K 38/14; C07H 5/00; C07H 5/10; A61K 38/16; A61P 17/02; C08B 37/04; A61K 47/36

(52) U.S. Cl. 514/8; 536/55.1; 536/54; 514/2; 536/3; 514/777; 514/12

(57) ABSTRACT

The invention relates to a polysaccharide comprising carboxyl functional groups, one at least of which is substituted by a derivative of a hydrophobic alcohol. The invention also relates to a pharmaceutical composition comprising one of the polysaccharides according to the invention and at least one active principle. It also relates to a pharmaceutical composition, wherein the active principle is chosen from the group consisting of proteins, glycoproteins, peptides and nonpeptide therapeutic molecules. The invention also relates to the use of the functionalized polysaccharides according to the invention in the preparation of pharmaceutical compositions as described above.

The present invention relates to novel biocompatible polymers based on polysaccharides comprising carboxyl functional groups which can be of use, in particular for the administration of active principle(s) (AP(s)) to man or animals with a therapeutic and/or prophylactic purpose.

Hydrophobic alcohols are of advantage in the formulation of pharmaceutical active principles, in particular because of their biocompatibility and their hydrophobic nature, which makes it possible to adjust the hydrophobicity of the polymers to which they may be grafted.

Their biocompatibility is excellent insofar as they play a role in many biochemical processes and are present in the esterified form in the majority of tissues.

However, it is known to a person skilled in the art that it is difficult to graft an alcohol to a polysaccharide comprising carboxyl functional groups since it is difficult to be selective between the hydroxyl functions of the polysaccharide and the hydroxyl function of the hydrophobic alcohol. During grafting, the alcohols of the polymer may compete with the alcohol of the graft, if it is not desired to have recourse to techniques for the protection/deprotection of the alcohols of the polymer, and this side reaction results in the crosslinking of the polymer chains. Thus, advantageous hydrophobic alcohols, such as cholesterol, could not to date be grafted to polysaccharides comprising carboxyl functional groups.

Dellacherie et al. have developed esters of polysaccharides, i.e. of alginates, of hyaluronates (Pelletier, S. et al., Carbohydr. Polym., 2000, 43, 343-349) or of galacturonans (Dellacherie, Edith et al., Langmuir, 2001, 17, 1384-1391), by a synthetic method employing alkyl α-halides, bromododecane and bromooctadecane. The synthesis of the esters consists in substituting the halides by tetrabutylammonium carboxylates. This method makes it possible to access esters of hydrophobic alcohols but it is limited to halogenated alkyl derivatives which can undergo nucleophilic substitution. It thus cannot be employed to graft hydrophobic alcohols such as cholesterol. Furthermore, these halogenated derivatives exhibit risks of toxicity and are thus not safe to use in the development of a pharmaceutical product.

Other researchers have got round this difficulty by grafting hydrophobic acids instead of hydrophobic alcohols. Nichifor et al., for example, have employed cholic acid, a steroid derivative, in order to graft it directly to dextran alcohols (Nichifor, Marieta et al., Eur. Polym. J., 1999, 35, 2125-2129). This method gets round the problem of cholesterol by employing a derivative exhibiting a carboxylic acid capable of reacting with the alcohols of a polysaccharide. However, cholic acid is not approved by the FDA for injections, in contrast to cholesterol, and this strategy cannot be employed with polysaccharides comprising carboxyl functional groups.

Other researchers have employed nonanionic polysaccharides in order to be able to graft hydrophobic alcohols. Akiyoshi et al., for example, have converted cholesterol, which is nucleophilic, to an electrophilic derivative (Biomacromolecules, 2007, 8, 2366-2373). This electrophilic derivative of cholesterol could be grafted to the alcohol functions of pullulan or mannan, which are neutral polysaccharides. Again, this strategy cannot be employed with polysaccharides comprising carboxyl functional groups.

A recent review of functional dextran-based polymers (Heinze, Thomas et al., Adv. Polym. Sci., 2006, 205, 199-291) reports modifications by hydrophobic acids, inter alia, but does not report dextran functionalized by hydrophobic alcohols.

The present invention relates to novel derivatives of amphiphilic polysaccharides comprising carboxyl functional groups partially substituted by at least one hydrophobic alcohol derivative. These novel derivatives of polysaccharides comprising carboxyl functional groups have a good biocompatibility and their hydrophobicity can be easily adjusted without detrimentally affecting the biocompatibility.

It also relates to a synthetic method which makes it possible to solve the abovementioned synthesis problems. This method has made it possible to obtain polysaccharides comprising carboxyl functional groups partially substituted by hydrophobic alcohols, including, for example, cholesterol.

The invention thus relates to polysaccharides comprising carboxyl functional groups, one at least of which is substituted by a derivative of a hydrophobic alcohol, denoted HA:
said hydrophobic alcohol (HA) being grafted or bonded to the anionic polysaccharide via a coupling arm R, said coupling arm being bonded to the anionic polysaccharide via a function F, said function F resulting from the coupling between the amine function of the connecting arm R and a carboxyl function of the anionic polysaccharide, and said coupling arm being bonded to the hydrophobic alcohol via a function G resulting from the coupling between a carboxyl, isocyanate, thioacid or alcohol function of the coupling arm and a function of the hydrophobic alcohol, the unsubstituted carboxyl functions of the anionic polysaccharide being in the cationic carboxylate form, the cation preferably being that of an alkali metal, such as Na⁺ or K⁺,
   F being an amide function,
   G being either an ester, thioester, carbonate or carbamate function,
   R being a chain comprising between 1 and 18 carbons which is optionally branched and/or unsaturated, which optionally comprises one or more heteroatoms, such as O, N and/or S, and which has at least one acid function,
HA being a residue of a hydrophobic alcohol, the product of the coupling between the hydroxyl function of the hydrophobic alcohol and at least one electrophilic function carried by the group R,
said polysaccharide comprising carboxyl functional groups being amphiphilic at neutral pH.

In one embodiment, G is an ester function.

According to the invention, the polysaccharide comprising carboxyl functional groups partially substituted by hydrophobic alcohols is chosen from the polysaccharides comprising carboxyl functional groups of general formula I:

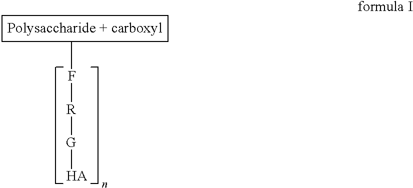

in which n represents the molar fraction of the carboxyl functions of the polysaccharide substituted by F-R-G-HA and is between 0.01 and 0.7,

F, R, G and HA corresponding to the definitions given above, and, when the carboxyl function of the polysaccharide is not substituted by F-R-G-HA, then the carboxyl functional group or groups of the polysaccharide are cation carboxylates, the cation preferably being that of an alkali metal, such as Na⁺ or K⁺.

In one embodiment, the polysaccharides comprising carboxyl functional groups are polysaccharides naturally carrying carboxyl functional groups and are chosen from the group consisting of alginate, hyaluronan and galacturonan.

In one embodiment, the polysaccharides comprising carboxyl functional groups are synthetic polysaccharides obtained from polysaccharides naturally comprising carboxyl functional groups or from neutral polysaccharides, to which at least 15 carboxyl functional groups per 100 saccharide units have been grafted, of general formula II:

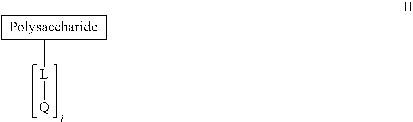

the natural polysaccharides being chosen from the group of polysaccharides predominantly composed of glycoside monomers bonded via glycoside bonds of (1,6) and/or (1,4) and/or (1,3) and/or (1,2) type,
L being a bond resulting from the coupling between the connecting arm Q and an —OH function of the polysaccharide and being either an ester, thioester, carbonate, carbamate or ether function,
i representing the molar fraction of the L-Q substituents per saccharide unit of the polysaccharide,
Q being a chain comprising between 1 and 18 carbons which is optionally branched and/or unsaturated, which comprises one or more heteroatoms, such as O, N and/or S, and which comprises at least one carboxyl functional group —CO₂H.

In one embodiment, n is between 0.05 and 0.5.

In one embodiment, the polysaccharide is predominantly composed of glycoside monomers bonded via glycoside bonds of (1,6) type.

In one embodiment, the polysaccharide predominantly composed of glycoside monomers bonded via glycoside bonds of (1,6) type is dextran.

In one embodiment, the polysaccharide is predominantly composed of glycoside monomers bonded via glycoside bonds of (1,4) type.

In one embodiment, the polysaccharide predominantly composed of glycoside monomers bonded via glycoside bonds of (1,4) type is chosen from the group consisting of pullulan, alginate, hyaluronan, xylan, galacturonan and a water-soluble cellulose.

In one embodiment, the polysaccharide is a pullulan.

In one embodiment, the polysaccharide is an alginate.

In one embodiment, the polysaccharide is a hyaluronan.

In one embodiment, the polysaccharide is a xylan.

In one embodiment, the polysaccharide is a galacturonan.

In one embodiment, the polysaccharide is a water-soluble cellulose.

In one embodiment, the polysaccharide is predominantly composed of glycoside monomers bonded via glycoside bonds of (1,3) type.

In one embodiment, the polysaccharide predominantly composed of glycoside monomers bonded via glycoside bonds of (1,3) type is a curdlan.

In one embodiment, the polysaccharide is predominantly composed of glycoside monomers bonded via glycoside bonds of (1,2) type.

In one embodiment, the polysaccharide predominantly composed of glycoside monomers bonded via glycoside bonds of (1,2) type is an inulin.

In one embodiment, the polysaccharide is predominantly composed of glycoside monomers bonded via glycoside bonds of (1,4) and (1,3) type.

In one embodiment, the polysaccharide predominantly composed of glycoside monomers bonded via glycoside bonds of (1,4) and (1,3) type is a glucan.

In one embodiment, the polysaccharide is predominantly composed of glycoside monomers bonded via glycoside bonds of (1,4) and (1,3) and (1,2) type.

In one embodiment, the polysaccharide predominantly composed of glycoside monomers bonded via glycoside bonds of (1,4) and (1,3) and (1,2) type is mannan.

In one embodiment, the group Q of the polysaccharide according to the invention is chosen from the following groups:

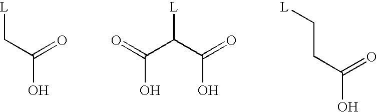

In one embodiment, i is between 0.1 and 2.

In one embodiment, i is between 0.2 and 1.5.

In one embodiment, the group R according to the invention is noteworthy in that it is chosen from amino acids.

In one embodiment, the amino acids are chosen from α-amino acids.

In one embodiment, the α-amino acids are chosen from natural α-amino acids.

In one embodiment, the natural α-amino acids are chosen from leucine, alanine, isoleucine, glycine, phenylalanine, tryptophan or valine.

In one embodiment, the hydrophobic alcohol is chosen from fatty alcohols.

In one embodiment, the hydrophobic alcohol is chosen from the alcohols composed of a saturated or unsaturated and branched or unbranched alkyl chain comprising from 4 to 18 carbons.

In one embodiment, the hydrophobic alcohol is chosen from the alcohols composed of a saturated or unsaturated and branched or unbranched alkyl chain comprising from 6 to 18 carbons.

In one embodiment, the hydrophobic alcohol is chosen from the alcohols composed of a saturated or unsaturated and branched or unbranched alkyl chain comprising from 8 to 16 carbons.

In one embodiment, the hydrophobic alcohol is octanol.

In one embodiment, the hydrophobic alcohol is 2-ethylbutanol.

In one embodiment, the fatty alcohol is chosen from myristyl alcohol, cetyl alcohol, stearyl alcohol, cetearyl alcohol, butyl alcohol, oleyl alcohol or lanolin.

In one embodiment, the hydrophobic alcohol is chosen from cholesterol derivatives.

In one embodiment, the cholesterol derivative is cholesterol.

In one embodiment, the hydrophobic alcohol is chosen from menthol derivatives.

In one embodiment, the hydrophobic alcohol is menthol in its racemic form.

In one embodiment, the hydrophobic alcohol is the D isomer of menthol.

In one embodiment, the hydrophobic alcohol is the L isomer of menthol.

In one embodiment, the hydrophobic alcohol is chosen from tocopherols.

In one embodiment, the tocopherol is α-tocopherol.

In one embodiment, the α-tocopherol is the racemate of α-tocopherol.

In one embodiment, the tocopherol is the D isomer of α-tocopherol.

In one embodiment, the tocopherol is the L isomer of α-tocopherol.

In one embodiment, the hydrophobic alcohol is chosen from alcohols carrying an aryl group.

In one embodiment, the alcohol carrying an aryl group is chosen from benzyl alcohol or phenethyl alcohol.

The polysaccharide can have a degree of polymerization m of between 10 and 10 000.

In one embodiment, it has a degree of polymerization m of between 10 and 1000.

In another embodiment, it has a degree of polymerization m of between 10 and 500.

The invention also relates to the synthesis of the polysaccharides comprising partially substituted carboxyl functional groups according to the invention.

Said synthesis comprises a step of obtaining an amino intermediate HA-G-R-NH₂ or an ammonium salt HA-G-R-NH₃⁺, the counterion of which is an anion chosen from halides, sulfates, sulfonates or carboxylates, and a step of grafting this amino intermediate to a carboxyl function of a polysaccharide, R, G and HA corresponding to the definitions given above.

In one embodiment, a step of functionalizing the polysaccharide with at least 15 carboxyl functional groups per 100 saccharide units is carried out by grafting compounds of formula Q-L′, L′ being an anhydride, halide, carboxylic acid, thioacid or isocyanate function, to at least 15 alcohol functions per 100 saccharide units of the polysaccharide, Q and L corresponding to the definitions given above.

In one embodiment, the amino intermediate of formula HA-G-R-NH₂ or HA-G-R-NH₃⁺ is obtained by reaction of a compound of formula G′-R-NH₂, G′ being a carboxylic acid, isocyanate, thioacid or alcohol function, with the alcohol function of the hydrophobic alcohol, R, G and HA corresponding to the definitions given above.

If necessary, in this step for obtaining the amino intermediate, use is made of the protection/deprotection techniques well known to a person skilled in the art of peptide synthesis.

Preferably, the step of grafting the amino intermediate to an acid function of the polysaccharide is carried out in an organic medium.

The invention also relates to the use of the functionalized polysaccharides according to the invention in the preparation of pharmaceutical compositions as described above.

The invention also relates to a pharmaceutical composition comprising one of the polysaccharides according to the invention as described above and at least one active principle.

The invention also relates to a pharmaceutical composition according to the invention as described above, wherein the active principle is chosen from the group consisting of proteins, glycoproteins, peptides and nonpeptide therapeutic molecules.

Active principle is understood to mean a product in the form of a single chemical entity or in the form of a combination having a physiological activity. Said active principle can be exogenous, that is to say that it is introduced by the composition according to the invention. It can also be endogenous, for example the growth factors which will be secreted in a wound during the first phase of healing and which can be retained on said wound by the composition according to the invention.

Depending on the pathologies targeted, it is intended for a local or systemic treatment.

In the case of local and systemic releases, the methods of administration envisaged are by the intravenous, subcutaneous, intradermal, transdermal, intramuscular, oral, nasal, vaginal, ocular, buccal or pulmonary route, and the like.

The pharmaceutical compositions according to the invention are either in the liquid form, in aqueous solution, or in the powder, implant or film form. They additionally comprise the conventional pharmaceutical excipients well known to a person skilled in the art.

Depending on the pathologies and methods of administration, the pharmaceutical compositions can advantageously comprise, in addition, excipients which make it possible to formulate them in the form of a gel, sponge, injectable solution, solution to be taken orally, lyophilized tablet, and the like.

The invention also relates to a pharmaceutical composition according to the invention as described above, which can be administered in the form of a stent, of a film or coating of implantable biomaterials, or of an implant.

### EXAMPLE 1

### Synthesis of Sodium Dextranmethylcarboxylate Modified by Cholesterol Leucinate

Cholesterol leucinate, para-toluenesulfonic acid salt, is obtained according to the process described in the patent (Kenji, M et al., U.S. Pat. No. 4,826,818)

8 g (i.e. 148 mmol of hydroxyl functions) of dextran with a weight-average molar mass of approximately 40 kg/mol (Fluka) are dissolved in water at 42 g/l. 15 ml of 10N NaOH (148 mmol of NaOH) are added to this solution. The mixture is brought to 35° C. and then 23 g (198 mmol) of sodium chloroacetate are added. The temperature of the reaction medium is brought to 60° C. at 0.5° C./min and then maintained at 60° C. for 100 minutes. The reaction medium is diluted with 200 ml of water, neutralized with acetic acid and purified by ultrafiltration through a 5 kD PES membrane against 6 volumes of water. The final solution is quantitatively determined by dry extract, in order to determine the concentration of polymer, and then quantitatively determined by acid/base titration in 50/50 (v/v) water/acetone, in order to determine the degree of substitution with methylcarboxylates.

According to the dry extract: [polymer]=31.5 mg/g

According to the acid/base titration: the degree of substitution of the hydroxyl functions by methylcarboxylate functions is 1.04 per saccharide unit.

The sodium dextranmethylcarboxylate solution is passed over a Purolite resin (anionic) in order to obtain the dextranmethylcarboxylic acid, which is subsequently lyophilized for 18 hours.

8 g of dextranmethylcarboxylic acid (37 mmol of methylcarboxylic acid functions) are dissolved in DMF at 45 g/l and then cooled to 0° C. 0.73 g of cholesterol leucinate, para-toluenesulfonic acid salt (1 mmol) is suspended in DMF at 100 g/l. 0.11 g of triethylamine (1 mmol) is subsequently added to this suspension. Once the polymer solution is at 0° C., 0.109 g (1 mmol) of NMM and 0.117 g (1 mmol) of EtOCOCl are subsequently added. After reaction for 10 min, the cholesterol leucinate suspension is added. The medium is subsequently maintained at 4° C. for 15 minutes. The medium is subsequently heated to 30° C. Once at 30° C., the medium is subsequently run into a 5 g/l solution of 3.76 g of NMM (37 mmol) with vigorous stirring. The solution is ultrafiltered through a 10 kD PES membrane against 10 volumes of 0.9% NaCl solution and then 5 volumes of water. The concentration of the polymer solution is determined by dry extract. A fraction of solution is lyophilized and analyzed by ¹H NMR in D₂O in order to determine the level of acid functions converted to cholesterol leucinate amide.

According to the dry extract: [modified polymer]=12.9 mg/g

According to the ¹H NMR: the molar fraction of the acids modified by the cholesterol leucinate per saccharide unit is 0.03.

### EXAMPLE 2

### Synthesis of Sodium Dextransuccinate Modified by Cholesterol Leucinate

Cholesterol leucinate, para-toluenesulfonic acid salt, is obtained according to the process described in the patent (Kenji, M et al., U.S. Pat. No. 4,826,818).

Sodium dextransuccinate is obtained from dextran 40 according to the method described in the paper by Sanchez-Chaves et al. (Sanchez-Chaves, Manuel et al., Polymer, 1998, 39 (13), 2751-2757). The level of acid functions per glycoside unit (i) is 1.46, according to the ¹H NMR in D₂O/NaOD.

The sodium dextransuccinate solution is passed over a Purolite resin (anionic) in order to obtain dextransuccinic acid, which is subsequently lyophilized for 18 hours.

7.1 g of dextransuccinic acid (23 mmol) are dissolved in DMF at 44 g/l. The solution is cooled to 0° C. 0.77 g of cholesterol leucinate, para-toluenesulfonic acid salt (1 mmol) is suspended in DMF at 100 g/l. 0.12 g of triethylamine (TEA) (1 mmol) is subsequently added to this suspension.

Once the polymer solution is at 0° C., 0.116 g (1 mmol) of NMM and 0.124 g (1 mmol) of EtOCOCl are subsequently added. After reacting for 10 min, the cholesterol leucinate suspension is added. The medium is subsequently maintained at 4° C. for 15 minutes. The medium is subsequently heated to 30° C. Once at 30° C., the medium is subsequently run into a 5 g/l solution of 3.39 g of NMM (33 mmol) with vigorous stirring. The solution is ultrafiltered through a 10 kD PES membrane against 10 volumes of 0.9% NaCl solution and then 5 volumes of water. The concentration of the polymer solution is determined by dry extract. A fraction of solution is lyophilized and analyzed by ¹H NMR in D₂O in order to determine the level of acid functional groups converted to cholesterol leucinate amide.

According to the dry extract: [modified polymer]=17.5 mg/g

According to the ¹H NMR: the molar fraction of the acids modified by the cholesterol leucinate per saccharide unit is 0.05.

### EXAMPLE 3

### Synthesis of Sodium Pullulansuccinate Modified by Cholesterol Leucinate

Cholesterol leucinate, para-toluenesulfonic acid salt, is obtained according to the process described in the patent (Kenji, M et al., U.S. Pat. No. 4,826,818).

10 g of pullulan with a weight-average molar mass of approximately 100 kg/mol (Fluka) are dissolved in DMSO at a concentration of 400 mg/g at 60° C. This solution is equilibrated at 40° C. and then two DMF solutions comprising 9.27 g of succinic anhydride (371 g/l) and 9.37 g of NMM (375 g/l) are added to the pullulan solution. The reaction time is 240 min, starting from the addition of the NMM solution. The solution thus obtained is diluted in 1 l of water and ultrafiltered through a 10 kD PES membrane against a 0.9% sodium chloride solution and then against doubly distilled water. The concentration of sodium pullulansuccinate in the final solution is determined by dry extract and the dry product is analyzed by ¹H NMR in D₂O/NaOD in order to determine the level of hydroxyl functions converted to succinic ester per saccharide unit.

According to the dry extract: [pullulansuccinate]=15.8 mg/g

According to the ¹H NMR: the molar fraction of the alcohols carrying a sodium succinate per saccharide unit is 1.35.

The sodium pullulansuccinate solution is acidified on a Purolite resin (anionic) and is then subsequently lyophilized for 18 hours.

5 g of pullulansuccinic acid are dissolved in DMF at 51 g/l. The solution is cooled to 0° C. 0.08 g of NMM and 0.08 g of EtOCOCl are subsequently added. After reacting for 10 min, a suspension comprising 0.51 g of cholesterol leucinate, para-toluenesulfonic acid (PTSA) salt, and 0.08 g of TEA in 5.1 ml of DMF is added. The grafting time is 20 min, after the introduction of the cholesterol derivative. The medium is subsequently heated to 30° C. and then run into an aqueous NMM solution (2.09 g at 5 mg/ml). The solution obtained is diluted by adding 100 ml of water and then diafiltered through a 10 kD PES membrane against a 0.9% sodium chloride solution and then against doubly distilled water. The concentration of sodium pullulansuccinate modified by the cholesterol leucinate in the final solution is determined by dry extract and the dry product is analyzed by ¹H NMR in D₂O/NaOD in order to determine the level of acid functions converted to cholesterol leucinate amide.

According to the dry extract: [modified polymer]=2.9 mg/g

According to the ¹H NMR: the molar fraction of the acids modified by the cholesterol leucinate per saccharide unit is 0.04.

### EXAMPLE 4

### Synthesis of Sodium Pullulansuccinate Modified by the Alaninate of Cetyl Alcohol

The alaninate of cetyl alcohol is obtained according to the process described in the patent (Kenji, M et al., U.S. Pat. No. 4,826,818).

A sodium pullulansuccinate solution obtained as described in example 3 is acidified on a Purolite resin (anionic) and is then subsequently lyophilized for 18 hours.

5 g of pullulansuccinic acid are dissolved in DMF at 51 g/l. The solution is cooled to 0° C. 0.32 g of NMM (3.2 mmol) and 0.32 g of EtOCOCl (3.2 mmol) are subsequently added. After reacting for 10 min, a suspension comprising 1.55 g of alaninate of cetyl alcohol, para-toluenesulfonic acid salt (3.2 mmol) and 0.32 g of TEA (3.2 mmol) in 20.4 ml of DMF is added. The grafting time is 20 min, after the introduction of the cetyl alcohol derivative. The medium is subsequently heated to 30° C. and then run into an aqueous NMM solution (8.36 g at 5 mg/ml). The solution obtained is diluted by adding 100 ml of water and then diafiltered through a 10 kD PES membrane against a 0.9% sodium chloride solution and then against doubly distilled water. The concentration of sodium pullulansuccinate modified by the alaninate of cetyl alcohol in the final solution is determined by dry extract and the dry product is analyzed by ¹H NMR in D₂O/NaOD in order to determine the level of acid functions converted to amide of alaninate of cetyl alcohol.

According to the dry extract: [modified polymer]=5.2 mg/g

According to the ¹H NMR: the molar fraction of the acids modified by alaninate of cetyl alcohol per saccharide unit is 0.18.

### EXAMPLE 5

### Synthesis of Sodium Dextranmethylcarboxylate Modified by Dodecanol Alaninate

Dodecanol alaninate, para-toluenesulfonic acid salt, is obtained according to the process described in the patent (Kenji, M et al., U.S. Pat. No. 4,826,818).

A sodium dextranmethylcarboxylate solution obtained as described in example 1 is passed over a Purolite resin (anionic) in order to obtain dextranmethylcarboxylic acid, which is subsequently lyophilized for 18 hours.

5 g of dextranmethylcarboxylic acid (23.2 mmol of methylcarboxylic acid functions) are dissolved in DMF at 45 g/l and then cooled to 0° C. 1.99 g of dodecanol alaninate, para-toluenesulfonic acid salt (4.6 mmol) are suspended in DMF at 100 g/l. 0.47 g of triethylamine (4.6 mmol) is subsequently added to this suspension. Once the polymer solution is at 0° C., 2.35 g (23.2 mmol) of NMM and 2.52 g (23.2 mmol) of EtOCOCl are subsequently added. After reacting for 10 min, the dodecanol alaninate suspension is added. The medium is subsequently maintained at 4° C. for 15 minutes. The medium is subsequently heated to 30° C. Once at 30° C., an imidazole solution (3.2 g in 9.3 ml of water) is added to the reaction medium. The polymer solution is ultrafiltered through a 10 kD PES membrane against 10 volumes of 0.9% NaCI solution and then 5 volumes of water. The concentration of the polymer solution is determined by dry extract. A fraction of solution is lyophilized and analyzed by ¹H NMR in D₂O in order to determine the level of acid functions modified by dodecanol alaninate.

According to the dry extract: [modified polymer]=22 mg/g

According to the ¹H NMR: the molar fraction of the acids modified by the dodecanol alaninate per saccharide unit is 0.19.

### EXAMPLE 6

### Synthesis of Sodium Dextranmethylcarboxylate Modified by L-Menthol Glycinate

L-Menthol glycinate, para-toluenesulfonic acid salt, is obtained according to the process described in the patent (Kenji, M et al., U.S. Pat. No. 4,826,818).

As the oil obtained comprises impurities, the amine salt is neutralized by a stoichiometric addition of sodium hydroxide and extracted with diisopropyl ether. The organic phase is then acidified with a solution of HCl in ethyl ether and the HCl salt of the menthol derivative is extracted with water. After lyophilization, L-menthol glycinate, hydrochloric acid salt, is obtained.

A sodium dextranmethylcarboxylate solution obtained as described in example 1 is passed over a Purolite resin (anionic) in order to obtain dextranmethylcarboxylic acid, which is subsequently lyophilized for 18 hours.

12 g of dextranmethylcarboxylic acid (59.22 mmol of methylcarboxylic acid functions) are dissolved in DMF at 60 g/l and then cooled to 0° C. 1.32 g of L-menthol glycinate, hydrochloric acid salt, (5.29 mmol) are suspended in DMF at 100 g/l. 0.54 g of triethylamine (5.29 mmol) is subsequently added to this suspension. Once the polymer solution is at 0° C., a solution of NMM (6.59 g, 65.1 mmol) in DMF (530 g/l) and 7.07 g (65.1 mmol) of EtOCOCl are subsequently added. After reacting for 10 minutes, the L-menthol glycinate suspension is added. The medium is subsequently maintained at 10° C. for 45 minutes. The medium is subsequently heated to 50° C. An imidazole solution (14.7 g in 22 ml of water) and 65 ml of water are added to the reaction medium. The polymer solution is ultrafiltered through a 10 kD PES membrane against 6 volumes of 0.9% NaCI solution, 4 volumes of 0.01N sodium hydroxide solution, 7 volumes of 0.9% NaCl solution and then 3 volumes of water. The concentration of the polymer solution is determined by dry extract. A fraction of solution is lyophilized and analyzed by ¹H NMR in D₂O in order to determine the level of acid functions converted to L-menthol glycinate amide.

According to the dry extract: [modified polymer]=25.7 mg/g

According to the ¹H NMR: the molar fraction of the acids modified by the L-menthol glycinate per saccharide unit is 0.09.

### EXAMPLE 7

### Synthesis of Sodium Dextranmethylcarboxylate Modified by (±)-α-Tocopherol Alaninate

(±)-α-Tocopherol alaninate, hydrochloric acid salt, is obtained according to the process described in J. Pharm. Sci., 1995, 84(1), 96-100.

A sodium dextranmethylcarboxylate modified by (±)-α-tocopherol alaninate is obtained by a process similar to that described in example 6.

According to the dry extract: [modified polymer]=28.1 mg/g

According to the ¹H NMR: the molar fraction of the acids modified by the (±)-α-tocopherol alaninate per saccharide unit is 0.04.

### EXAMPLE 8

### Synthesis of Sodium Dextranmethylcarboxylate Modified by Octanol Glycinate

Octanol glycinate, para-toluenesulfonic acid salt, is obtained according to the process described in the patent (Kenji, M et al., U.S. Pat. No. 4,826,818).

A sodium dextranmethylcarboxylate modified by octanol glycinate is obtained by a process similar to that described in example 6.

According to the dry extract: [modified polymer]=34.1 mg/g

According to the ¹H NMR: the molar fraction of the acids modified by the octanol glycinate per saccharide unit is 0.27.

### EXAMPLE 9

### Synthesis of Sodium Dextranmethylcarboxylate Modified by Octanol Phenylalaninate

Octanol phenylalaninate, para-toluenesulfonic acid salt, is obtained according to the process described in the patent (Kenji, M et al., U.S. Pat. No. 4,826,818).

A sodium dextranmethylcarboxylate modified by octanol phenylalaninate is obtained by a process similar to that described in example 6.

According to the dry extract: [modified polymer]=30.2 mg/g

According to the ¹H NMR: the molar fraction of the acids modified by the octanol phenylalaninate per saccharide unit is 0.09.

### EXAMPLE 10

### Synthesis of Sodium Dextranmethylcarboxylate Modified by the Phenylalaninate of Benzyl Alcohol

A sodium dextranmethylcarboxylate modified by the phenylalaninate of benzyl alcohol is obtained, by a process similar to that described in example 6, using the phenylalaninate of benzyl alcohol, hydrochloric acid salt (Bachem).

According to the dry extract: [modified polymer]=47.7 mg/g

According to the ¹H NMR: the molar fraction of the acids modified by the phenylalaninate of benzyl alcohol per saccharide unit is 0.41.

### EXAMPLE 11

### Synthesis of Sodium Dextranmethylcarboxylate Modified by Isohexanol Phenylalaninate

Isohexanol phenylalaninate, para-toluenesulfonic acid salt, is obtained according to the process described in the patent (Kenji, M et al., U.S. Pat. No. 4,826,818).

A sodium dextranmethylcarboxylate modified by isohexanol phenylalaninate is obtained by a process similar to that described in example 6.

According to the dry extract: [modified polymer]=29.8 mg/g

According to the ¹H NMR: the molar fraction of the acids modified by the isohexanol phenylalaninate per saccharide unit is 0.18.

### EXAMPLE 12

### Dissolution of a BMP-2 Lyophilizate

A test of dissolution of a Bone Morphogenetic Protein 2 (BMP-2) lyophilizate was developed in order to demonstrate the solubilizing power of different polymers as a physiological pH. The BMP-2 is dissolved in a buffer comprising sucrose (Sigma), glycine (Sigma), glutamic acid (Sigma), sodium chloride (Riedel-de-Haën) and polysorbate 80 (Fluka). The pH of this solution is adjusted to pH 4.5 by addition of sodium hydroxide and then the solution is lyophilized. 283.2 mg of lyophilizate comprise approximately 12 mg of BMP-2.

The polymers according to the invention are employed in this test. Sodium dextranmethylcarboxylate modified by ethyl phenylalaninate, a polymer described in patent application FR0702316, is also employed in this test by way of comparison.

The test consists in introducing approximately exactly 4 mg of lyophilizate comprising 0.168 mg of BMP-2. The lyophilizate is subsequently taken up in 210 μl of an aqueous solution in order to achieve a final concentration of BMP-2 to 0.8 mg/ml as a physiological pH, the final concentration of polymer being 5 mg/ml.

The visual appearance of the solution is recorded after stirring for 5 minutes at a low speed on a roll.

The results for different solutions are collated in the following table.

| Solution | Visual appearance | pH |
|---|---|---|
| Water | clear | 4.3 |
| Example 9 | clear | 7.4 |
| Example 8 | clear | 7.5 |
| Example 5 | clear | 7.4 |
| Counterexample FR0702316 | cloudy | 7.5 |

The addition of water results in a clear BMP-2 solution but at an acidic pH

This test makes it possible to demonstrate the improvement in the dissolution of BMP-2 at a physiological pH by the polymers according to the invention. On the other hand, sodium dextranmethylcarboxylate modified by ethyl phenylalaninate does not make it possible to obtain a clear BMP-2 solution.

1. A polysaccharide comprising carboxyl functional groups, one at least of which is substituted by a derivative of a hydrophobic alcohol, denoted HA:
said alcohol being grafted or bonded to the anionic polysaccharide via a coupling arm R, said coupling arm being bonded to the anionic polysaccharide via a functional group F, said function F resulting from the coupling between the amine function of the connecting arm R and a carboxyl function of the anionic polysaccharide, and said coupling arm being bonded to the hydrophobic alcohol via a function G resulting from the coupling between a carboxyl, isocyanate, thioacid or alcohol function of the coupling arm and a function of the hydrophobic alcohol, the unsubstituted carboxyl functions of the anionic polysaccharide being in the cationic carboxylate form, the cation preferably being that of an alkali metal, such as Na+ or K+,
F being an amide function,
G being either an ester, thioester, carbonate or carbamate function,
R being a chain comprising between 1 and 18 carbons which is optionally branched and/or unsaturated, which optionally comprises one or more heteroatoms, such as O, N and/or S, and which has at least one acid function,
HA being a residue of a hydrophobic alcohol, the product of the coupling between the hydroxyl function of the hydrophobic alcohol and at least one electrophilic function carried by the group R,
said polysaccharide comprising carboxyl functional groups being amphiphilic at neutral pH.

2. The polysaccharide as claimed in claim 1, which is chosen from polysaccharides comprising carboxyl functional groups, at least one of which is substituted by a derivative of a hydrophobic alcohol, of general formula I: 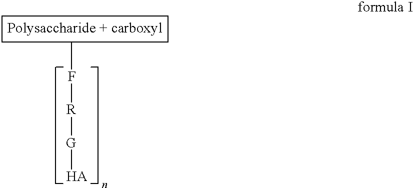
in which n represents the molar fraction of the carboxyl functions of the polysaccharide substituted by F R G HA and is between 0.01 and 0.7,
F, R, G and HA corresponding to the definitions given above, and, when one or more carboxyl functions of the polysaccharide are not substituted by F R G HA, then the carboxyl function or functions of the polysaccharide are cation carboxylates, the cation preferably being that of an alkali metal, such as Na+ or K+.

3. The polysaccharide as claimed in claim 1, the anionic polysaccharide naturally carrying acid functions and being chosen from the group consisting of alginate, hyaluronan and galacturonan.

4. The polysaccharide as claimed claim 1, the anionic polysaccharide being a synthetic anionic polysaccharide obtained from an anionic or nonanionic (neutral) natural polysaccharide to which at least one acid function has been grafted, of general formula II: 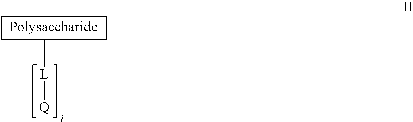
the natural polysaccharide being chosen from the group of the polysaccharides predominantly composed of glycoside monomers bonded via glycoside bonds of (1,6) and/or (1,4) and/or (1,3) and/or (1,2) type,
L being a bond resulting from the coupling between the connecting arm Q and an —OH function of the neutral or anionic polysaccharide, being either an ester, thioester, carbonate, carbamate or ether function,
Q being a chain comprising between 1 and 18 carbons which is optionally branched and/or unsaturated, which comprises one or more heteroatoms, such as O, N and/or S, and which comprises at least one acid function CO₂H.

5. The polysaccharide as claimed in claim 4, the polysaccharide being predominantly composed of glycoside monomers bonded via glycoside bonds of (1,6) type.

6. The polysaccharide as claimed in claim 5, the polysaccharide predominantly composed of glycoside monomers bonded via glycoside bonds of (1,6) type being dextran.

7. The polysaccharide as claimed in claim 4, the polysaccharide being predominantly composed of glycoside monomers bonded via glycoside bonds of (1,4) type.

8. The polysaccharide as claimed in claim 7, the polysaccharide predominantly composed of glycoside monomers bonded via glycoside bonds of (1,4) type being chosen from the group consisting of pullulan, alginate, hyaluronan, xylan, galacturonan or a water-soluble cellulose.

9. The polysaccharide as claimed in claim 4, the polysaccharide being predominantly composed of glycoside monomers bonded via glycoside bonds of (1,3) type.

10. The polysaccharide as claimed in claim 9, the polysaccharide predominantly composed of glycoside monomers bonded via glycoside bonds of (1,3) type being a curdlan.

11. The polysaccharide as claimed in claim 4, the polysaccharide being predominantly composed of glycoside monomers bonded via glycoside bonds of (1,2) type.

12. The polysaccharide as claimed in claim 11, the polysaccharide predominantly composed of glycoside monomers bonded via glycoside bonds of (1,2) type being an inulin.

13. The polysaccharide as claimed in claim 4, the polysaccharide being predominantly composed of glycoside monomers bonded via glycoside bonds of (1,4) and (1,3) type.

14. The polysaccharide as claimed in claim 13, the polysaccharide predominantly composed of glycoside monomers bonded via glycoside bonds of (1,4) and (1,3) type being a glucan.

15. The polysaccharide as claimed in claim 4, the polysaccharide being predominantly composed of glycoside monomers bonded via glycoside bonds of (1,4) and (1,3) and (1,2) type.

16. The polysaccharide as claimed in claim 15, the polysaccharide predominantly composed of glycoside monomers bonded via glycoside bonds of (1,4) and (1,3) and (1,2) type being mannan.

17. The polysaccharide as claimed in claim 4, wherein the group Q is chosen from the following groups:

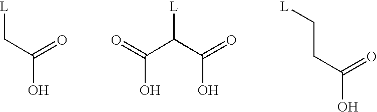

18. The polysaccharide as claimed in claim 1, wherein the group R is chosen from amino acids.

19. The polysaccharide as claimed in claim 1, wherein the group R is chosen from a amino acids.

20. The polysaccharide as claimed in claim 19, the a amino acids being chosen from natural a amino acids, including leucine, alanine, isoleucine, glycine, phenylalanine, tryptophan or valine.

21. The polysaccharide as claimed in claim 1, the hydrophobic alcohol being chosen from fatty alcohols.

22. The polysaccharide as claimed in claim 21, the hydrophobic alcohol being chosen from the alcohols composed of a saturated or unsaturated and branched or unbranched alkyl chain comprising from 4 to 18 carbons.

23. The polysaccharide as claimed in claim 1, the fatty alcohol being chosen from myristyl alcohol, cetyl alcohol, stearyl alcohol, cetearyl alcohol, butyl alcohol, oleyl alcohol or lanolin.

24. The polysaccharide as claimed in claim 21, the hydrophobic alcohol being cholesterol.

25. The polysaccharide as claimed in claim 21, the hydrophobic alcohol being menthol.

26. The polysaccharide as claimed in claim 21, the hydrophobic alcohol being chosen from tocopherols, preferably a tocopherol.

27. The polysaccharide as claimed in claim 21, the hydrophobic alcohol being chosen from alcohols carrying an aryl group.

28. The polysaccharide as claimed in claim 27, the alcohol carrying an aryl group being chosen from benzyl alcohol or phenethyl alcohol.

29. The use of a functionalized polysaccharide as claimed in claim 1 in the preparation of pharmaceutical compositions as described above.

30. A pharmaceutical composition comprising a polysaccharide as claimed in claim 1 and at least one active principle.

31. The pharmaceutical composition as claimed in claim 30, which can be administered by the oral, nasal, vaginal or buccal route.

32. The pharmaceutical composition as claimed in claim 30, wherein the active principle is chosen from the group consisting of proteins, glycoproteins, peptides and nonpeptide therapeutic molecules.
